(19) United States
(12) Patent Application Publication
(10) Pub. No.: US 2013/0231732 A1
(43) Pub. Date: Sep. 5, 2013

(54) Implantable Graft Assembly and Aneurysm Treatment

(71) Applicants: Carlos Vonderwalde, Richmond (CA); Carlos Gonzalez-Resendiz, Richmond (CA)

(72) Inventors: Carlos Vonderwalde, Richmond (CA); Carlos Gonzalez-Resendiz, Richmond (CA)

(73) Assignee: DESIGN & PERFORMANCE - CYPRUS LIMITED, Nicosia (KY)

(21) Appl. No.: 13/868,286

(22) Filed: Apr. 23, 2013

Related U.S. Application Data
(62) Division of application No. 12/160,234, filed on Jul. 8, 2008.
(60) Provisional application No. 60/764,760, filed on Feb. 3, 2006.

(51) Int. Cl. A61F 2/95

(52) U.S. Cl. CPC A61F 2/95
USPC 623/1.13

(57) ABSTRACT

Disclosed is a method of treating an aneurysm by deploying an implantable graft assembly.

### RELATED APPLICATIONS

This application is a Divisional application of U.S. Ser. No. 12/160,234 published as US 2009/0069880, a US National Phase Application of PCT/IL2007/000140 having an International Filing Date of Feb. 4, 2007, that claims priority from U.S. Provisional Patent Application 60/764,760 filed Feb. 3, 2006. The three applications are incorporated by reference in their entirety.

### FIELD AND BACKGROUND OF THE INVENTION

The present invention relates to the field of medicine, and more particularly to the field of intracorporeal implantable devices especially implantable graft assemblies. The present invention also relates to the treatments of aneurysms, especially cerebral aneurysms and aneurysms of bifurcated vessels.

An aneurysm is a localized ballooning of a blood vessel by more than 50% of the diameter of the vessel. Aneurysms can occur in any blood vessel, although they are most common in arteries, particularly in the arteries at the base of the brain (the Circle of Willis) and in the aorta. Approximately 85% of cerebral aneurysms develop in the anterior part of the Circle of Willis and involve the internal carotid arteries and major branches thereof. The most common sites include the anterior communicating artery (30-35%), the bifurcation of the internal carotid and posterior communicating artery (30-35%), the bifurcation of the middle cerebral artery (20%), the bifurcation of the basilar artery, and the remaining posterior circulation arteries (5%).

Rupture of an aneurysm causes severe pain, internal hemorrhage, and, without prompt treatment, may result in death. In addition the aneurysm may split, which may block vessels that branch from the vessel on which the aneurysm is located, or may release blood clots which block the flow of blood to other regions of the body.

A cerebral aneurysm may result, for example, from trauma, infection, or neoplastic disease. Most cerebral aneurysms, however, result from a developmental abnormality of an arterial lining together with abnormal thinning of the arterial wall. It appears there may be a genetic predisposition to the development of cerebral aneurysms. External factors which are believed to contribute to the formation of cerebral aneurysms include cigarette smoking, traumatic head injury, excess alcohol consumption and the use of oral contraceptives. The existence of cerebral aneurysms is associated with other diseases such as polycystic kidney disease, coarctation of the aorta, and fibromuscular hyperplasia. It is estimated that up to one in 15 people in the United States will develop a cerebral aneurysm during their lifetime.

Rupture of a cerebral aneurysm causes bleeding into the subarachnoid space surrounding the brain, causing a subarachnoid hemorrhage (SAH). The bleeding may irritate, damage or destroy nearby brain cells, resulting in brain damage with paralysis or coma. In severe cases, the bleeding may lead to death. Blood from a ruptured aneurysm can block circulation of cerebrospinal fluid (CSF), leading to fluid buildup and increased pressure on the brain. Brain ventricles may become enlarged, resulting in hydrocephalus. The bleeding may also lead to vasospasm, wherein the blood vessels of the brain narrow, such that insufficient blood is supplied to the brain, and a stroke may result.

The main goals of treatment of an unruptured aneurysm are to prevent rupture or growth of the aneurysm. The main goals of treatment of a ruptured aneurysm are to stop the bleeding and to prevent or limit vasospasm, as well as to reduce the risk of recurrence.

Aortic aneurysms are commonly treated by surgical repair of the aorta, typically involving insertion of a synthetic patch tube. Alternatively, treatment may involve placement of a covered stent via a percutaneous technique into the diseased portion of the aorta to seal the aneurysm. Percutaneous stenting of aortic aneurysms has a lower mortality rate than an open surgical approach.

Stents are usually outwardly radially expandable, having a substantially tubular shape both in an unexpanded state with a small radial dimension and in any one of the expanded states with larger radial dimensions. Various constructions of stents are known including rolled-up sheets, slotted or otherwise cut-out tubes and bent wires.

For deployment inside a lumen of a bodily vessel an expandable stent is placed in an unexpanded state on a deployment catheter, inserted through an incision in the skin and maneuvered through the body to the deployment location. The stent is then expanded to an appropriately-sized expanded state so as to engage the inner walls of the treated vessel and to thus maintain patency thereof.

A first type of stent is the self-expanding stent. When the stent is at the deployment location, the stent is released from the catheter and allowed to expand to an expanded state, in a manner analogous to that of a compressed spring. Self-expanding stents have been disclosed, for example, in U.S. Pat. Nos. 4,503,569; 4,580,568; 4,787,899; and 5,104,399.

A second type of stent is expanded from the unexpanded state to an expanded state using an expansion device, typically a catheter-borne balloon. When the stent is at the deployment location, the expansion device is activated inside the bore of the unexpanded stent to exert an outwards radial force on the inside of the stent, causing the stent to expand to a desired size. Such stents have been disclosed, for example, in U.S. Pat. Nos. 4,655,771; 4,733,665; 4,739,762; 4,800,882; 4,907,336; 4,994,071; 5,019,090; 5,035,706; 5,037,392; and 5,147,385.

Conventional stents are open-walled and if used in degenerated SVG (Saphenous Vein Grafts) allow material protrusion to pass between the structural elements that define the stent body such as struts and in native arteries when a side branch is involved, permit the flow of blood through the stent walls. Covered stents are stent assemblies made up of a stent with a stent cover (also called a jacket), a sheet of synthetic or polymeric material or biological tissue, the stent cover closing the openings in the stent walls are known. It is known to deploy covered stents to treat an aortic aneurysm: the covered stent is deployed so that the stent cover closes off the aneurysm neck.

As is known to one skilled in the art, many blood vessels of the body are bifurcated. By “bifurcated” is meant an object that splits to two branches along a length of the object, and generally comprises a trunk vessel from which a branch vessel branches at a bifurcation point. An aneurysm may be situated on or near a bifurcation point. A problem with using a covered stent to treat an aneurysm on such a bifurcated vessel is that the stent cover may partially or totally obstruct the entrance into the branch vessel, stopping flow into the branch vessel, increasing pressure at the bifurcation point and causing turbulent flow, factors that may lead to stenosis of the trunk vessel or of the branch vessel or damage to parts of the body dependent on blood from the branch vessel.

It would be highly advantageous to have a covered stent useful for deployment in bifurcated vessels, especially where a branch vessel is involved, for the treatment of aneurysms, devoid of at least some of the disadvantages of the prior art.

Invasive and non-invasive surgical treatments for cerebral aneurysms are known.

Invasive surgical techniques involve removal of a section of the skull (craniotomy) to access the aneurysm, followed by clipping of the aneurysm with a metal clip. Such invasive surgical methods are prone to complications and are associated with high patient mortality.

Endovascular coiling of cerebral aneurysms is a minimally invasive surgical procedure that accesses the treatment area from within the affected blood vessel with a catheter, to provide partial or complete treatment of aneurysms. A typical endovascular coiling procedure involves insertion of a catheter into the femoral artery in the patient's leg, which is navigated through the vascular system, into the head and to proximity with the aneurysm. Flexible platinum coils are threaded through the catheter and deployed inside the aneurysm, blocking blood flow into the aneurysm and thus preventing rupture. Endovascular coiling can be performed under general anesthesia or light sedation. A disadvantage of endovascular coiling is the length of time and cost required for the procedure since large aneurysms may require the use of several individual coils.

An alternative treatment, particularly for large aneurysms, involves filling the aneurysm with a liquid embolic material (e.g., cellulose acetate polymer), which solidifies to occlude the aneurysm. A disadvantage of this method is that the embolic material may leak into the arterial lumen.

In the treatment of wide-neck aneurysms, both endovascular coiling and liquid filling techniques require the use of a stent to retain the coils or the embolic material, respectively, inside the aneurysm. However, the majority of currently available stents are not suitable for cerebrovascular applications, being too rigid and not sufficiently flexible to negotiate the inherently tortuous vascularity of the intracranial circulation and have a large, relative to the cerebral vasculature, unexpanded diameter.

The use of intracranial stent-assisted coil placement in the treatment of unruptured, wide-necked cerebral aneurysms has been disclosed. Jabbour et al. Neurosurg. Focus 17(5): 1-4, 2004; and Sani et al., Neurosurg. Focus 18(2): 1-5, 2005, using the Neuroform Microstent™, which is an uncovered, flexible, self-expanding nitinol stent. The initial treatment stage in stent-assisted coil placement involves deployment of the stent in the artery across the aneurysm neck using a first microcatheter of up to 1 mm (3 French) outer diameter. A second microcatheter is navigated between the struts of the stent into the aneurysm sac. The aneurysm sac is then filled with coils or embolic material through the second microcatheter. The stent works as a scaffold to prevent the coils or embolic material from migrating out of the aneurysm sac through the aneurysm neck. This procedure is time-consuming and costly.

It would be desirable to treat cerebral aneurysms by sealing the aneurysm in a single-step procedure involving the use of a covered stent. The use of covered stents for treatment of aneurysms in the brain has been precluded: the addition of a cover to a stent reduces the stent flexibility and increases the outer diameter of the stent, see for example “Stent-Graft Placement for Wide-Neck Aneurysm of the Vertebrobasilar Junction” by M. A. Burbelkoa; L. A. Dzyakb; N. A. Zorinb; S. P. Grigorukc; and V. A. Golykb.

In the art it is known to cover stents with covers made of serous membrane, see U.S. Pat. Nos. 6,254,627 and 6,468,300 of the.

Serous membrane is a type of tissue that holds various organs together and include the peritoneum (the serous membrane that lines the cavity of the abdomen of a mammal and is folded inward over the abdominal and pelvic viscera), the pericardium (the conical sac of serous membrane that encloses the heart and the roots of the great blood vessels), the pleura (the serous membrane that lines each half of the thorax and is folded back over the surface of the lung of the same side) and dura mater (the tough serous membrane covering the brain and the spinal cord and lining the inner surface of the skull). The serous membranes release a lubricating serous fluid allowing the expanding and contracting organs (including the lungs) held within a given serous membrane to slide gently against adjacent parts of the body.

Serous membranes are made of two strata. The serous stratum of a serous membrane is a very smooth single layer of flattened, nucleated mesothelial cells united at their edges by cement substance that secrete the lubricating serous fluid. The serous stratum is the side that faces towards and contacts the organs. The serous cells rest on a basement layer, a rough, strong fibrous layer that forms a protective sack about the serous stratum. Beneath the basement layer are networks of yellow elastic and white fibers imbedded in a ground substance that also contains connective-tissue cells.

The use of serous membranes as a component of intracorporeal implants is known in the art.

In U.S. Pat. No. 4,502,159 is taught a method for preparing a tubular graft from pericardium.

In U.S. Pat. No. 5,782,914 is taught a method for processing animal tissue such as serous membranes for use as a graft material in intracorporeal implants.

In U.S. Pat. No. 5,934,283 is taught the use of tissues, including serous membranes such as pericardium, peritoneum and tunica vaginalis in fashioning a pubovaginal sling.

In U.S. Pat. No. 5,865,723 and in PCT Patent Application No. PCT/US96/20868 published as WO 97/24081 is discussed that vascular prostheses of autologous pericardial membrane fashioned into tubular grafts have been used but have been proven to be ineffective, for example as the pericardial membrane is subject to rupture and structural failure. Therefore in WO 97/24081 is taught a stent assembly comprising pericardial, fascial rectus sheath or venous tissue formed as a cover over a stent. The tissue is harvested, usually but not necessarily treated in a stabilizing medium, and attached to the outside of the stent by rolling over the stent so that the two edges of the tissue overlap by at least 35° (and preferably are wrapped twice about the stent) so as to obviate the need for sutures.

Due to the overlapping layers of pericardium, stent assemblies covered in accordance with the teachings of WO 97/24081 are quite thick, causing a significant reduction in the bore size of a bodily vessel in which deployed, limiting such stent assemblies for deployment only to relatively large bore lumina. Further, the thickness of a stent cover made in accordance with the teachings of WO 97/24081 reduces the flexibility and consequently maneuverability of such a stent assembly, limiting the locations in which such covered stents can be deployed.

In PCT Patent Application No. PCT/US96/13907 published as WO 97/09006 is taught a stent assembly comprising a cover of at least one layer of pericardium (preferably human, bovine or porcine origin) covering at least a portion of the inside or outside surface of a stent. The thickness of the cover is adjusted by varying the number of layers of pericardium. The disadvantages of such stent assemblies are similar to those of WO 97/24081.

The prior art does not disclose a covered stent which is suitable for intracranial use. It would therefore be highly advantageous to have a covered stent or such device which is suitable for deployment within the brain, particularly for use in the treatment of cerebral aneurysms and for the treatment of cerebral aneurysm in proximity of a bifurcation.

### SUMMARY OF THE INVENTION

Embodiments of the present invention successfully address at least some of the shortcomings of prior art by providing implantable graft assemblies exceptionally useful for the treatement of aneurysms, especially such aneurysms as cerebral aneurysms, wide-necked aneurysms or aneurysms of bifurcated vessels. Embodiments of the present invention allow for substantial sealing of the neck of an aneurysm on a bifurcated vessel by providing a relatively small graft, that due its small size causes little or no blockage or interference with branch. Embodiments of the present invention provide an implantable graft assembly that has a lower profile and is more flexible due to the small size of the graft, allowing maneuvering through smaller vessels such as found in the brain with less fear of damage.

Thus, according to the teachings of the present invention there is provided an implantable graft assembly comprising a graft secured to a radially expandable substantially tubular frame, wherein in an expanded state a first portion of the surface area of the frame is covered by the graft and a second portion of the surface area of the frame is free of the graft, the first portion having a circumferential section which is less than the entire circumference of the frame constituting no more than about 95%, no more than about 80%, no more than about 67%, no more than about 50% and even no more than about 34% of the surface area of the frame.

According to the teachings of the present invention there is also provided a method of treating an aneurysm, comprising: a) providing an implantable graft assembly (such as described above) comprising a graft secured to a radially expandable substantially tubular frame, wherein in an expanded state a first portion of the surface area of the frame is covered by the graft and a second portion of the surface area of the frame is free of the graft, the first portion constituting no more than about 95% of the surface area of the frame; b) providing a delivery system for deploying the implantable graft assembly within a blood vessel on which the aneurysm is located; and c) deploying the implantable graft assembly within the blood vessel using the delivery system, such that the portion of the frame covered by the graft is positioned across a neck of the aneurysm. In embodiments of the present invention the graft assembly is self-expanding. In embodiments of the present invention the graft assembly is not self-expanding and the delivery system comprises an expansion device, for example a balloon catheter.

In embodiments of the present invention, the first portion has a length substantially the same as the length of the frame

In embodiments of the present invention, the first portion has a length less than about 75%, less than about 50% and even less than about 34% of the total length of the frame.

In embodiments of the present invention, the first portion covers a circumferential section which is less than the entire circumference of the frame. In embodiments of the present invention, the first portion covers a circumferential section not more than about 330°, not more than about 270°, not more than about 240°, not more than about 180°, not more than about 120° or even covering not more than about 90° of the entire circumference of the frame.

In embodiments of the present invention, the tubular frame is configured to be self-expanding (e.g., analogous to self-expanding stents known in the art). In embodiments of the present invention, the tubular frame is configured to radially expand by application of an outwards force applied to an inner surface of the tubular frame (e.g., analogous to balloon expandable stents known in the art), for example as applied by a standard catheter-mounted balloon.

In embodiments of the present invention, the tubular frame is substantially a stent. In embodiments of the present invention, the stent comprises at least three radially expandable ring sections, each the ring section connected to a neighboring ring section with at least one longitudinally arrayed strut, each ring section having an outer surface and an inner surface, wherein the graft covers, at least partially, an outer surface of at least one the ring sections and the graft is disposed under and makes contact with an inner surface of at least one other the ring section. In embodiments of the present invention, a first end of the graft is disposed under and makes contact with an inner surface of a first the ring section, a second end of the graft is disposed under and makes contact with an inner surface of a second the ring section, and a portion of the graft between the ends cover, at least partially, an outer surface of at least one the ring section located between the first and the second ring sections. Such embodiments are preferably implemented with stents such as the “Over and Under” stent by Design & Performance (Cyprus) Ltd or a stent such as described in U.S. Pat. No. 6,699,277 of the Inventor. Specifically, when the teachings of the present invention are implemented using a stent such as described in U.S. Pat. No. 6,699,277, the graft is placed in a manner substantially analogous to the described therein, substantially as depicted in FIG. 1D or FIG. 1E.

In embodiments of the present invention, the substantially tubular frame comprises two radially-expandable ring sections longitudinally spaced apart and mutually connected with at least two longitudinally arrayed struts arrayed about a cirumference of each the ring section, for example, as disclosed in PCT/IB012/00315 published as WO 01/66037 of the Inventor.

In embodiments, along the length of at least one (and preferably at least two) of the longitudinally arrayed struts are a plurality of graft-connecting features to which the graft is secured, for example with the help of sutures. In embodiments the graft-connecting features are integrally formed with the struts. In embodiments, the graft-connecting features are separate components secured to the struts, for example by welding. In embodiments, the graft-connecting features are related to the shape of the struts, e.g., the struts are sinusoidal, zigzag and the like (for example, as disclosed in PCT/IB012/00315 published as WO 01/66037 of the Inventor) and the graft-connecting features are the minima and maxima of the shape. In embodiments, the features are distinct features such as open eyelets, closed eyelets, open loops, closed loops and the like, distributed along the length of a strut. In embodiments, a strut is a substantially perforated band or strip, each perforation constituting a graft-connecting feature.

In embodiments, at least one (preferably more than one) longitudinally arrayed strut is a component of a graft-connecting feature configured to secure the graft to the frame by clamping. In embodiments, at least part of the longitudinally arrayed strut is of a temperature dependent shape-memory material where at low temperatures the strut is in a non-clamping configuration and at higher temperatures is in a clamping configuration, e.g. non-clamping at about 0° C. or lower and clamping at about 20° C. or higher. In embodiments, at least one (preferably more than one) longitudinally arrayed strut comprises two longitudinally arrayed elastic members each attached at a first end to a first radially-expandable ring section, attached at a second end to a second radially-expandable ring section and biased towards each other thereby being configured to clamp the graft, so that the graft is clamped between the two longitudinally arrayed elastic members.

In embodiments of the present invention, the ring sections are mutually connected with at least four longitudinally arrayed struts.

In general, it is preferred that no two struts be disposed at 180° from each other on the circumference of the radially expandable ring sections so as not to limit the axial flexibility of the frame. As is it generally preferred that such devices be symmetrical, in embodiments of the present invention the ring sections are mutually connected with an odd number of longitudinally arrayed struts, for example at least three or even at least five longitudinally arrayed struts.

In embodiments of the present invention the graft is substantially sheet-like (including, for example, planar or somewhat curved sheets). Preferably, to reduce the profile and to increase axial flexibility it is preferred that the graft be relatively thin. In embodiments of the present invention, the thickness of the graft is up to about 0.45 mm, up to about 0.2 mm and even up to about 0.11 mm.

In embodiments of the present invention, the length of the graft is substantially equal to the length of the frame. In embodiments of the present invention, the length of the graft is substantially shorter than the length of the frame.

In embodiments of the present invention the graft has two ends defining a length of the graft and two edges defining a width of the graft. In embodiments, the graft is substantially rectangular. In embodiments the graft is associated (e.g. attached or secured) with the frame in such a way that the two edges abut or overlap so that when deployed the graft covers an entire circumference of the frame. In embodiments of the present invention, the width of the graft is less than a circumference of the frame in an expanded state and the graft is associated (e.g., attached or secured) with the frame in such a way that between the two edges is a gap, generally through which is apparent a portion of the frame.

In embodiments of the present invention, an end of the graft is secured to an end of the frame. In embodiments of the present invention, both ends of the graft are secured to an end of the frame.

As discussed above, in embodiments of the present invention the frame comprises two radially expandable ring sections mutually connected with at least two longitudinally arrayed struts. In embodiments, both ends of the graft are secured each to a ring section. In embodiments, one end of the graft is secured to a ring section. In embodiments, an end of the graft is secured to at least two struts, preferably to graft-connecting features on the struts. In embodiments, both ends of the graft are secured each to at least two of the struts, preferably to graft-connecting features on the struts. In embodiments, each of the two edges of the graft are secured to a strut, preferably to graft-connecting features on the struts.

In embodiments of the present invention, the graft comprises a synthetic or polymeric material, including but not limited to polytetrafluoroethylene, urethane, elastomer, polyamide and polyester.

In embodiments of the present invention, the graft comprises a biological material, including but not limited to a biological material (including but not limited to material from a human source, an equine source, a porcine source or a bovine source.) selected from the group consisting autologous tissue, heterologous tissue, venous tissue, arterial tissue, serous tissue, dura mater, pleura, peritoneum, pericardium and aortic leaflet. In preferred embodiments, the graft comprises serous tissue, especially serous membrane devoid of at least a portion of associated basement tissue, and even devoid of all the associated basement tissue to substantially comprise only a serous stratum. The use of serous tissue and serous membranes in implantable assemblies is taught in U.S. Pat. Nos. 6,254,627 and 6,468,300 of the Inventor.

In embodiments of the present invention, the graft is substantially disposed on an outer surface of the frame. In embodiments of the present invention, the graft is substantially disposed on an inner surface of the frame.

In embodiments of the present invention, the graft is of a material substantially impermeable to fluids.

In embodiments of the present invention, the graft is of a material substantially impervious to cell profileration therethrough.

In embodiments of the present invention, the graft is of a material allowing profileration of cells therethrough. In such embodiments, the graft constitutes a lining prosthesis which ultimately repairs the blood vessel in which deployed.

In embodiments of the present invention, the graft is of a stretchable material. In embodiments of the present invention, the graft is of a collapsible material, allowing folding of the graft for deployment.

In embodiments of the present invention, the graft is secured to the frame with at least one member of the group consisting of sutures, hooks, piercing members, clamps, adhesives, staples and bending members.

In embodiments of the present invention, a therapeutic or diagnostic agent releasably contained within the frame, the graft, or both.

In embodiments of the present invention, the graft includes at least one marker (e.g., functionally associated with the graft, the frame or both) detectable by a medical imaging modality, such as radiation emission, X-ray transmission, magnetic resonance imaging or ultrasound. The marker or markers allow the orientation and position of the graft to be accurately ascertained during deployment. In embodiments, at least one marker is disposed on at least one border selected from the group consisting of ends and an edges of the graft. In embodiments, the marker or marker delineate the edges and/or the ends of the graft.

In embodiments of the present invention, there is an alignment hole penetrating through the graft, preferably positioned substantially near the center of the graft. Preferably the alignment hole is small, having a diameter of no more than about 1 mm, no more than about 0.5 mm and even no more than about 0.376 mm. In embodiments, the alignment hole is about 0.35 mm. In embodiments, the alignment hole is reinforced, for example being encircled by a grommet or such component. Materials from which to make such a grommet include, but are not limited to biological tissue such as muscle tissue, polymers such as silicon rubber or metals such as stainless steel, nitinol, gold and titanium. In embodiments, such a grommet constitutes a marker as described above.

In embodiments of the method of the present invention, the delivery system is configured to control the radial orientation of the graft within the blood vessel, exceptionally useful in embodiments where the graft covers a circumferential section which is less than the entire circumference of the frame.

In embodiments, the delivery system is configured to control the radial orientation of the graft by rotation of the implantable graft assembly when inside the body during the deployment process. In embodiments, the delivery system comprises a delivery catheter on which the implantable graft assembly is positionable and a catheter guiding guide wire, and deploying comprises:

i) placing the catheter guiding guide wire in the blood vessel across the neck of the aneurysm;

ii) maneuvering the implantable graft assembly to proximity of the neck of the aneurysm on the delivery catheter along the catheter guiding guide wire;

iii) rotating the implantable graft assembly so that the graft is located across the neck of the aneurysm; and

iv) expanding the tubular frame

thereby pressing the graft against walls of the blood vessel and over the neck of the aneurysm so as to substantially seal the neck of the aneurysm. In embodiments, rotating is with reference to an observable marker (functionally associated with, for example, the graft, the frame and/or the delivery system), for example a marker observable by a medical imaging modality. Subsequently, the delivery system is withdrawn.

In embodiments, the delivery system is configured to control radial orientation of the graft with reference to an orientation guide wire. In embodiments, the delivery system comprises a catheter guiding guide wire, an orientation guide wire and a delivery catheter, the delivery catheter including:

a region near a distal end of the delivery catheter on which the implantable graft assembly is positionable for deployment;

a first lumen for engaging the catheter guiding guide wire running from a proximal end of the delivery catheter through a distal end of the delivery catheter;

a second lumen for engaging the orientation guide wire running from a proximal end of the delivery catheter through a port in the side of the delivery catheter proximal to the region on which the implantable graft assembly is positionable

and deploying the assembly comprises:

i) placing the implantable graft assembly on the region of the delivery catheter so that the graft is in line with the port of the second lumen;

ii) placing the catheter guiding guide wire in the blood vessel across the neck of the aneurysm;

iii) placing the orientation guide wire in the blood vessel and entering the aneurysm through the neck of the aneurysm;

iv) mounting the delivery catheter onto the guide wires, the catheter guiding guide wire in the first lumen and the orientation guide wire in the second lumen and through the port;

v) maneuvering the implantable graft assembly to proximity of the neck of the aneurysm along the catheter guiding guide wire and the orientation guide wire, thereby ensuring that the graft is aligned with the neck of the aneurysm; and

vi) expanding the tubular frame;

thereby pressing the graft against walls of the blood vessel and across the neck of the aneurysm so as to substantially seal the neck of the aneurysm. In such embodiments, for example, the catheter guiding guide wire passes through the entire delivery catheter from outside the patient all the way through the end of the delivery catheter, including through the region of the delivery catheter over which the implantable graft assembly is mounted, analogously to guide wires known in the art of stent delivery. In such embodiments, the fact that the orientation guide wire passes through the delivery catheter and emerges through a port proximally to the region of the delivery catheter over which the implantable graft assembly is located and is mounted on the same side where the graft is positioned and into the aneurysm through the neck of the aneurysm means that as the delivery catheter with the orientation guide wire is pushed along the two guide wires, the entire delivery catheter is directed in such a way that the graft is properly located with respect to the neck of the aneurysm.

In embodiments, during iv, the orientation guide wire is placed on the outside of the graft; and the orientation guide wire is withdrawn from the aneurysm prior to expanding of the tubular frame so as not to interfere with the expansion.

In embodiments, during iv the orientation guide wire is placed through an alignment hole penetrating through the graft. In such embodiments, the orientation guide wire may be withdrawn from the aneurysm prior to or subsequent to expanding of the tubular frame.

In embodiments of the method of the present invention, the blood vessel is bifurcated. Preferably, during deployment the portion of the frame that is free of the graft is positioned at a bifurcation of the bifurcated blood vessel so as not to obstruct with flow (e.g., of blood) between the trunk and branch vessels of the bifurcated vessel.

In embodiments of the method of the present invention, the aneurysm is a cerebral aneurysm.

In embodiments of the method of the present invention, the aneurysm is a wide-necked aneurysm.

According to the teachings of the present invention, there is also provided a radially expandable substantially tubular frame useful as a component of an implantable graft assembly comprising: a) at least two radially-expandable ring sections longitudinally spaced apart; and b) where the at least two ring sections are mutually connected with at least two longitudinally arrayed struts arrayed about a cirumference of each ring section, at least one longitudinally arrayed strut comprising two longitudinally arrayed elastic members each attached at a first end to a first radially-expandable ring section, attached at a second end to a second radially-expandable ring section and biased towards each other, thereby configured to clamp, for example a tissue or graft, therebetween. In embodiments, at least part of at least one longitudinally arrayed elastic member is of a temperature dependent shape-memory material where at low temperatures the two elastic members are spread apart in a non-clamping configuration and at higher temperatures the two elastic members are close together in a clamping configuration.

According to the teachings of the present invention there is also provided a method of making an implantable graft assembly (as described above); comprising: a) providing a radially expandable substantially tubular frame; b) providing a substantially sheet-like graft; and c) associating (e.g., securing or attaching) the graft to the frame so that the graft covers a first portion of the surface area of the frame, wherein the first portion has a circumferential section which is less than the entire circumference of the frame. In embodiments, the graft has two ends defining a length of the graft and two edges defining a width of the graft. In embodiments the graft is associated (e.g. attached or secured) with the frame in such a way that two edges of the graft abut or overlap so that when deployed the graft covers an entire circumference of the frame. In embodiments, the graft is associated (e.g., attached or secured) with the frame in such a way that between the two edges is a gap, generally through which is apparent a portion of the frame.

In embodiments, the tubular frame is substantially an expandable stent wherein, in embodiments, distributed over the surface of the frame are a plurality of graft-connecting features and wherein associating the graft to the frame comprises associating (e.g., securing or attaching) the graft to at least one graft-connecting feature in accordance with dimensions of the graft.

In embodiments, the substantially tubular frame comprises two radially-expandable ring sections mutually connected with at least two longitudinally arrayed struts arrayed about a cirumference of each ring section and wherein associating the graft to the frame comprises associating (e.g., securing or attaching) the graft to locations selected from the group consisting of the ring sections, the struts and a combination thereof. In embodiments, along the length of at least two of the longitudinally arrayed struts are a plurality of graft-connecting features and wherein the securing the graft to the frame comprises associating (e.g., securing or attaching) the graft to at least one graft-connecting feature in accordance with dimensions of the graft.

According to the teachings of the present invention there is also provided for the use of an implantable graft assembly as described above in the treatment of an aneurysm, especially an aneurysm located on a branched blood vessel, or a cerebral aneurysm, or a wide-necked aneurysm.

One of the greatest barriers to the use of covered stents and other graft-supporting expandable implants is that known grafts limit the flexibility of the implant and increase the unexpanded profile of the implant to a degree that makes deployment in the cerebrovascular implant difficult if not impossible. It has been found that a graft of serous tissue or membrane is strong enough, flexible enough and thin enough to overcome these problems. Thus, according to the teachings of the present invention there is also provided for the use of serous tissue (including, but not limited to, serous membrane devoid of at least a portion of associated basement layer and preferably devoid of all the basement layer to be substantially a serous stratum) for the preparation of a cerebrovascular implant, especially as a graft making up the implant, especially when the graft comprises a component of an expandable cerebrovascular implantable graft assembly.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1A, 1B, 1C, 1D and 1E depict graft assemblies useful in implementing the teachings of the present invention including an expandable frame that is substantially a stent;

FIGS. 2A, 2B, 2C, 2D, 2E and 2F depict graft assemblies useful in implementing the teachings of the present invention including an expandable frame that is substantially two terminal ring sections joined by longitudinal struts;

FIG. 3A is a depiction of a bifurcated artery with an aneurysm on the trunk vessel in which a graft assembly including a graft shorter than the frame is deployed;

FIG. 3B is a depiction of a bifurcated artery with an aneurysm on the trunk vessel in which a graft assembly including a graft not covering the entire circumference of the frame is deployed;

FIGS. 4A and 4B depict components of a delivery system allowing in vivo rotation of a graft assembly attached thereto;

FIG. 5 depicts components of a delivery system having an orientation guide wire that emerges from the catheter proximally to the graft assembly; and

FIGS. 6A and 6B depict components of a delivery system having an orientation guide wire that emerges from an alignment hole in the graft of the graft assembly.

### DESCRIPTION OF EMBODIMENTS OF THE INVENTION

Aspects of the present invention relate to implantable graft assembly and methods of using an implantable graft assembly, in embodiments exceptionally useful for deployment in cranial blood vessels (even with side branches involvement) and in bifurcated bodily vessels, especially for the treatment of aneurysms.

The principles, uses and implementations of the teachings of the present invention may be better understood with reference to the accompanying description and figures. Upon perusal of the description and figures present herein, one skilled in the art is able to implement the teachings of the present invention without undue effort or experimentation. In the figures, like reference numerals refer to like parts throughout.

Before explaining at least one embodiment of the invention in detail, it is to be understood that the invention is not limited in its application to the details set forth herein. The invention can be implemented with other embodiments and can be practiced or carried out in various ways. It is also understood that the phraseology and terminology employed herein is for descriptive purpose and should not be regarded as limiting.

Generally, the nomenclature used herein and the laboratory procedures utilized in the present invention include techniques from the fields of medicine, biology, chemistry, material sciences and engineering. Such techniques are thoroughly explained in the literature. Unless otherwise defined, all technical and scientific terms used herein have the same meaning as commonly understood by one of ordinary skill in the art to which the invention belongs. In addition, the descriptions, materials, methods and examples are illustrative only and not intended to be limiting. Methods and materials similar or equivalent to those described herein can be used in the practice or testing of the present invention. All publications, patent applications, patents and other references mentioned are incorporated by reference in their entirety as if fully set forth herein. In case of conflict, the specification herein will control.

As used herein, the terms “comprising” and “including” or grammatical variants thereof are to be taken as specifying the stated features, integers, steps or components but do not preclude the addition of one or more additional features, integers, steps, components or groups thereof. This term encompasses the terms “consisting of” and “consisting essentially of”.

The phrase “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed composition, device or method.

Herein the terms “jacket”, “graft”, and “cover” may, in some instances, be used interchangeably.

As noted in the introduction above, it is known to deploy a stent in the cerebrovascular system to hold coils inside of a cerebral aneurysm. It is known to treat aneurysms using a stent assembly comprising a stent and an associated graft. It is not known to treat a cerebral aneurysm using such a stent assembly: the graft about the stent causes the stent assembly to be too rigid and insufficiently flexible to be maneuvered through the cerebral vasculature. Further, such a stent assembly is too thick (has a large profile) to enter many cranial arteries. Further, as the cerebrovascular system is highly branched, there is the fear that the graft will block a necessary branch of the cerebrovascular system with catastrophic results.

It has been found that serous membrane, especially thinned serous membrane that is less than about 0.45 mm thick such as described in U.S. patent application Ser. No. 11/181,978 published as US 2005/0251244 of the Inventor, is thin enough, flexible enough and resilient enough to allow deployment of a stent assembly including a graft fashioned therefrom in the cerebrovascular system. Thus, an aspect of the present invention is the deployment of a stent assembly comprising a stent and a graft inside the cerebrovascular system, for example, for treating a cerebral aneurysm. Further, the aspect of the present invention is the use of serous membrane in the manufacture of a cerebral implant, such as a stent assembly for implantation in the brain for the treatment of cranial aneurysms.

An aspect of the present invention is related to implantable graft assemblies, exceptionally useful for deployment in cranial blood vessels or in bifurcated bodily vessels, comprising a graft secured to a radially expandable (self-expanding or, alternatively, configured to radially expand by application of an outwards force applied to an inner surface of the frame, such as, for example, by a balloon catheter) substantially tubular frame, wherein in an expanded state a first portion of the surface area of said frame is covered by the graft and a second portion of the surface area of the frame is free of the graft.

The implantable graft assemblies of the present invention are useful for the treatment of aneurysms, particularly aneurysms which are situated on a bifurcated blood vessel, and especially aneurysms of the cerebrovascular system. Such aneurysms are not amenable to the use of standard covered stents, since such would potentially block the flow of blood into branches leading off the stented artery, resulting in severe clinical consequences such as stroke when the artery is a cranial artery. When deployed within a blood vessel on which an aneurysm is situated, the implantable graft assembly of the present invention seals off the neck of the aneurysm, thereby preventing rupture or growth of an unruptured aneurysm, or further bleeding of a ruptured aneurysm. For treatment of an aneurysm located on a bifurcated blood vessel, the implantable graft of the present invention may be positioned so as to seal the neck of the aneurysm, without blocking blood vessels branching off the stented vessel.

Two important parameters used when selecting or designing an expandable frame for use as the frame of the present invention are the expanded and unexpanded diameters of the frame. Generally it is important that the unexpanded diameter of a frame be as small as possible to ease navigation through the bodily lumen to the deployment location. That said, the unexpanded diameter must be large enough to allow threading of the frame onto a delivery catheter and, if necessary, a frame-expanding device such as a stent-expanding balloon. Generally, any given frame has a wide range of expanded diameters. The expanded diameter of a frame subsequent to deployment is determined by the user of the graft assembly according to medical criteria including the natural size of the lumen of the vessel in which the graft assembly is deployed.

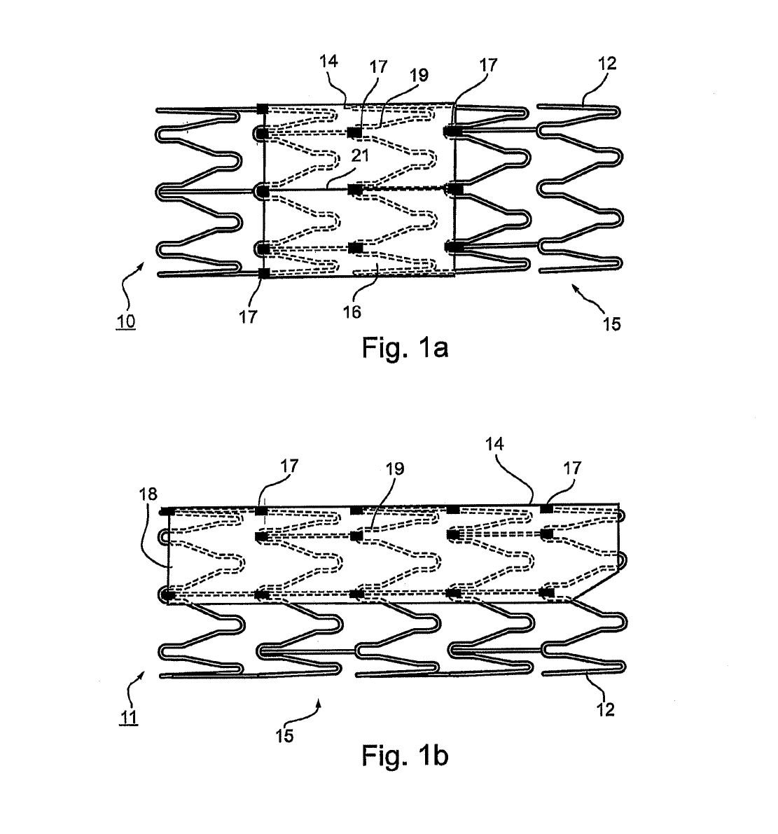

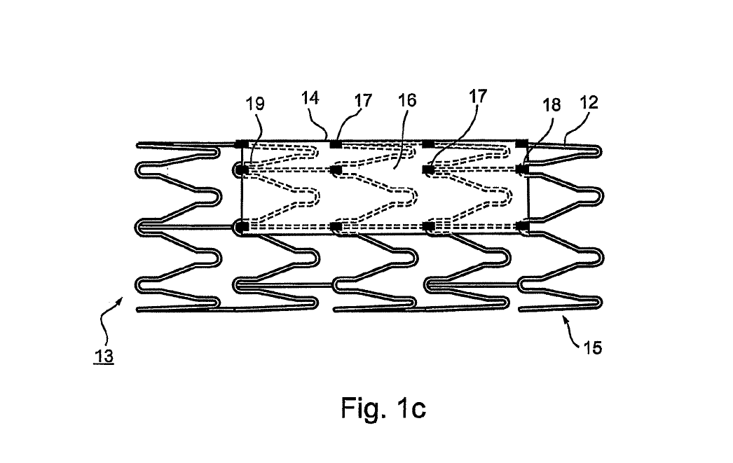

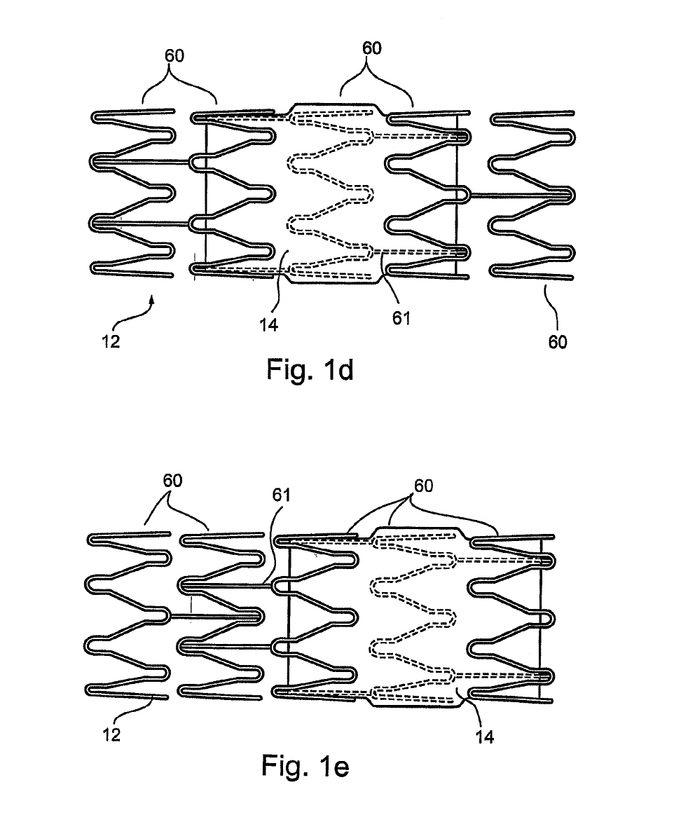

FIGS. 1A, 1B and 1C depict embodiments 10, 11 and 13 of the implantable graft assembly of the present invention, each comprising a graft 14 secured to a radially-expandable, substantially tubular frame 12 (in FIGS. 1A, 1B and 1C, a stent). The dimensions of a graft 14 are such that when frame 12 is fully expanded, implantable graft assemblies 10, 11 or 13 comprise both portions which are covered by grafts 14, and portions 15 which remain uncovered by graft 14, such that the area covered by graft 14 constitutes no more than about 95%, or no more than about 80%, or no more than about 67%, or no more than about 50%, or no more than about 34% of the surface area of frame 12. The fact that only a fraction of the surface area of frame 12 is covered by a graft 14 leads to a more flexible graft assembly.

In FIGS. 1A, 1B and 1C it is seen how grafts 14 are associated with frames 12 with the help of sutures 17 to a plurality of graft-connecting features 19, open eyelets which are an integral part of the ring sections making up frames 12. In embodiments, instead of sutures, a graft 14 is secured to a frame 12 with the help of other suitable components such as hooks, piercing members, clamps, adhesives, staples, tacks, pins and bending members, or other applicable mechanical mean or combinations thereof. In FIG. 1A is also seen seam 21 where the edges of the sheet making up graft 14 abut.

As depicted in FIGS. 1A and 1C for graft assemblies 10 and 13, the portion of frame 12 which is covered by graft 14 may be a longitudinal section 16 which is less than about 80% of the total length of frame 12. Optionally, according to this embodiment, a graft 14 is less than about 75%, or less than about 67%, or less than about 50%, or no more than about 34% of the total length of frame 12. Graft 14 may extend around the full circumference of section 16 to cover the entire outer surface of section 16, as depicted in FIG. 1A.

Alternatively, as shown in FIGS. 1B and 1C for graft assemblies 11 and 13, graft 14 may cover a circumferential section 18 (e.g., a partial arc) of frame 12 which is less than the entire circumference of frame 12, such that graft 14 forms a strip extending along the entire length of frame 12 (in the case of graft assembly 11) or part of the length of frame 12 (in the case of graft assembly 13). Optionally, according to this embodiment, graft 14 covers no more than about 330°, no more than about 270°, or no more than about 240°, or no more than about 180°, or no more than about 120°, or no more than about 90° of the entire circumference of frame 12.

For use in vessels other than those of the brain, generally any type of stent known in the art is useful as the frame of this embodiment of the implantable graft assembly of the present invention. Such stents include but are not limited to stents marketed by affiliates (e.g., Cordis, Centocor) of Johnson & Johnson, Guidant (Indianapolis, Ind., USA, now an affiliate of Boston Scientific Corp.), Medtronic (Minneapolis, Minn., USA), Medinol (Tel Aviv, Israel), Cook Inc. (Bloomington, Ind., USA) and Design & Performance (Cyprus) Ltd. (Cyprus). For deployment within intracranial blood vessels, a particularly thin, flexible stent such as the Over and Under™ stent (Design & Performance (Cyprus) Ltd.) is used, which is a premounted, low-pressure balloon-expandable stent constructed from an electro-polished stainless steel laser cut tube but also stents such as Neuroform stent (Boston Scientific Corp. Natick, Mass., USA), Neurolink stent (Guidant, Indianapolis, Ind., USA, now an affiliate of Boston Scientific Corp.) or Boa stent (Balt, Montmorency, France).

Further alternatively, as depicted in FIGS. 1D and 1E, frame 12 may comprise at least three radially expandable ring sections 60, with each ring section 60 being connected to a neighboring ring section 60 by at least one longitudinal strut 61. According to this embodiment of the present invention, graft 14 covers, at least partially, an outer surface of at least one ring section 60 and disposed under and makes contact with an inner surface of at least one other ring section 60. Optionally, a first end of graft 14 is disposed under and makes contact with an inner surface of a first ring section 60, and a second end of graft 14 is disposed under and makes contact with an inner surface of a second ring section 60, and a portion between the ends of graft 14 covers, at least partially, an outer surface of at least one ring section located between the first and second ring sections. Embodiments of stent assemblies such as the depicted in FIGS. 1D and 1E are taught in U.S. Pat. No. 6,699,277 of the Inventor.

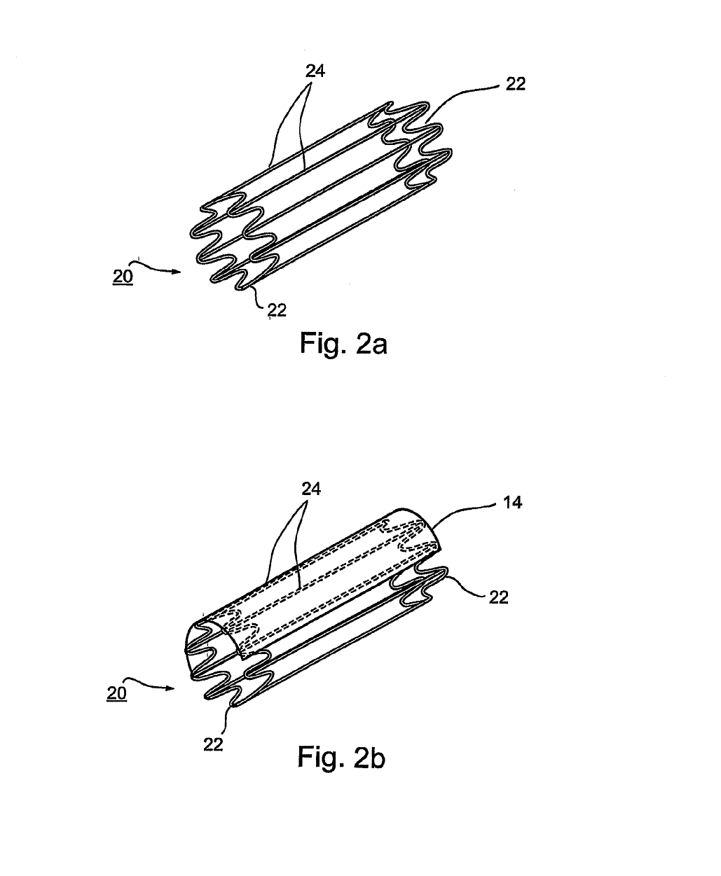

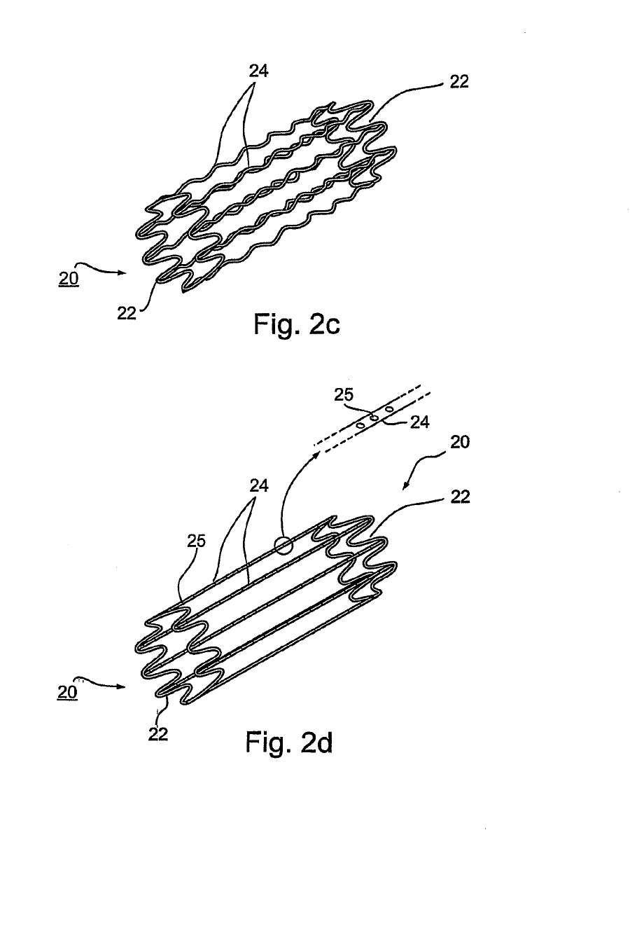

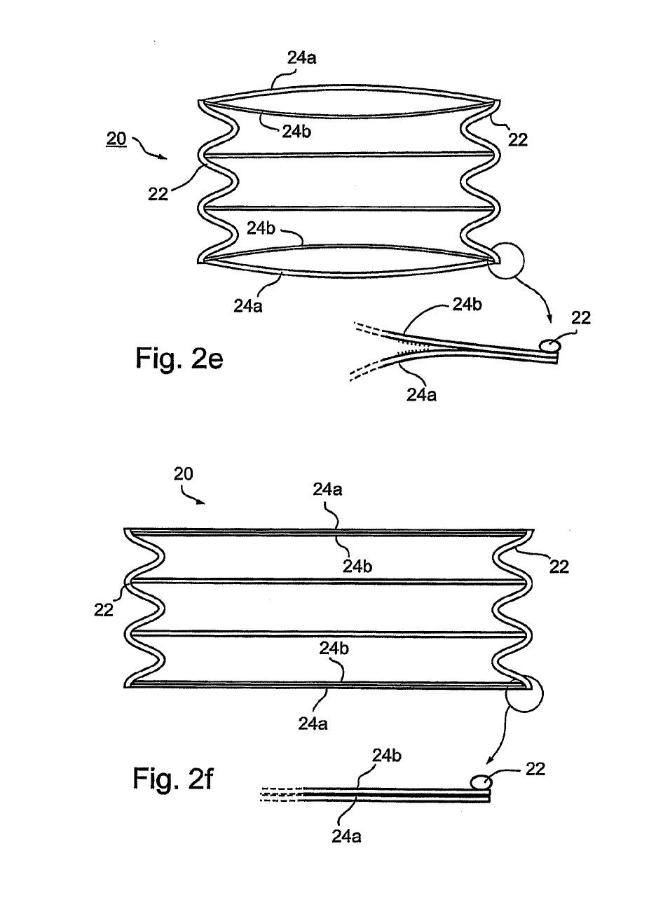

In an alternative embodiment of the present invention depicted in FIGS. 2A, 2B, 2C, 2D, 2E and 2F, frame 20 comprises at least terminal two radially-expandable ring sections 22, between which extend at least two longitudinal struts 24, arrayed about a circumference of each of ring sections 22. It is important to note that for clarity, only in FIG. 2B is a graft assembly depicted fully assembled as a graft 14 with a frame 20. while in FIGS. 2A, 2C, 2D, 2E and 2F frames 20 are depicted devoid of a graft 14. Ring sections 22 are optionally connected with at least four longitudinal struts 24. Struts 24 are optionally arrayed about the circumference of each of ring sections 22 such that no two adjacent struts are disposed at 180° from each other. Ring sections 22 are optionally connected by an odd number of longitudinal struts 24, for example three or five struts.

For example, in FIGS. 2A, 2B, 2C, 2D, 2E and 2F ring sections 22 are connected by eight struts 24, each strut separated by approximately 45° from a neighboring strut.

In embodiments, for example as depicted in FIG. 2B, graft is substantially sheet-like. Optionally, one or both ends of graft 14 may be secured to a ring section 22. Alternatively or additionally, one or both ends of graft 14 may be secured to at least two of the struts. Preferably, a sheet-like graft 14 is secured to two struts and spanned over one or more struts located between the two struts.

Ring sections 22 of a frame such as depicted in FIGS. 2A, 2B, 2C, 2D, 2E and 2F are preferably provided in a convoluted formation, such that the convolutions straighten to a certain degree upon expansion of frame 20 to provide an expanded frame diameter. Frame 20 has the advantage of providing a minimal interference to blood flow, since the total surface area of the frame is significantly less than that of a conventional stent frame. Furthermore, frames such as frames 20 are extremely flexible.

As depicted in FIG. 2B, implantable graft assembly 26 may comprise a graft 14 which extends around a portion of the circumference of frame 20 which is less than the entire circumference of frame 20, such that graft 14 forms a strip extending along substantially the entire length of frame 20. Optionally, according to this embodiment, graft 14 covers not more than about 270°, not more than about 270°, or not more than about 240°, or not more than about 180°, or not more than about 120°, or not more than about 90° of the entire circumference of frame 20.

Depicted in FIG. 2B is that graft 14 covers substantially the entire length of frame 20. The portion of frame 20 which is covered by graft 14 may alternatively be a longitudinal section which is less than about 75%, or less than about 67%, or less than about 50%, or less than about 34% of the total length of frame 20. In embodiments, graft 14 extends around the full circumference of frame 20 to cover the entire outer surface of the partial longitudinal section (not shown), analogously to the depicted in FIG. 1D. In embodiments, graft 14 covers a longitudinal section which is less than the total length of stent 20, and which extends around part of the circumference of stent 20 (not shown), analogously to the depicted in FIG. 1C.

According to any of the embodiments of the present invention, graft 14 is secured to the frame by any suitable means of attachment, such as, for example, sutures, hooks, piercing members, clamps, adhesives, staples, tacks, pins and bending members, or other applicable mechanical mean or combinations thereof. Optionally, graft 14 is secured to an end of the frame with suitable means of attachments.

In embodiments such as depicted in FIGS. 2A and 2B, struts 24 are substantially featureless flexible rods or wires, as disclosed in the PCT patent application published as WO 01/66037 of the Inventor.

In embodiments, along the length of at least one, preferably at least two, of the struts 24 are a plurality of graft-connecting features to which a graft 14 can be secured, for example, with the help of suitable attachment component such as sutures, hooks, piercing members, clamps, adhesives, staples and bending members, or other applicable mechanical mean. In FIG. 2C, the graft-connecting features are related to the wavy sinusoidal shape of struts 24 (for example, as disclosed in the PCT patent application published as WO 01/66037 of the Inventor). In FIG. 2D, struts 24 are perforated strips provided with a plurality of holes 25 as graft-connecting features. A graft 14 may be secured to a frame 20 of FIG. 2D with the help of suitable attaching components threaded through holes 25.

FIGS. 2E and 2F depict two different states of a same embodiment of a frame 20 made up of two expandable ring sections 22 made of wire (0.053″/1.35 mm diameter) where each of struts 24 is made up of two elastic members 24a and 24b of shape-memory alloy wire (0.025″/0.64 mm diameter). Elastic members 24a and 24b are laser welded at each end to each other and to a ring 22 thus securing the two ring sections 22 to each other in a longitudinally spaced apart fashion. In embodiments, a frame 20 such as depicted in FIGS. 2E and 2F is fashioned from a single laser-cut nitinol tube.

Elastic members 24a and 24b are configured so that at low temperatures (e.g., 0° C. and lower) these bow outwards so that a gap is formed there between, as depicted in FIG. 2E, and that at higher temperatures (e.g., 20° C. and higher) these straighten out or even bow inwards so that the gap closes, as depicted in FIG. 2F. In such a way, each of struts 24 constitutes a temperature dependent clamp where respective elastic members 24a and 24b are substantially jaws of the clamp. For use, a graft is placed between a pair of elastic members 24a and 24b in an open state (FIG. 2E) and the elastic members 24a and 24b allowed to heat and close to a clamping state (FIG. 2F), thereby clamping the graft there between. In FIGS. 2E and 2F, elastic members 24a and 24b are depicted as including features (serrations) on the facing (inner) faces to increase friction with a graft clamped therebetween.

The graft, the frame, or both, of any of the embodiments of the present invention may optionally further comprise a therapeutic or diagnostic agent releasably contained within the graft and/or the frame.

The graft of any of the embodiments of the present invention optionally further comprises an alignment hole penetrating through the graft, which may be in the center of the graft. Preferably, the alignment hole has a diameter of no more than about 1 mm, no more than about 0.5 mm, no more than about 0.0.376 mm. In embodiments, the alignment hole is about 0.35 mm. The alignment hole is optionally reinforced by a grommet, made, for example, of a material such as biological tissue, muscle tissue, polymer, silicon rubber, metal, gold and titanium. As will described below, in embodiments of the method of the present invention, an alignment hole is useful in directing the graft to the proper location to block the neck of the aneurysm by allowing a guide wire to pass through the graft into the aneurysm.

According to any of the embodiments of the present invention, a graft 14 may be substantially sheet-like, preferably substantially rectangular. The length of sheet-like graft 14 may optionally be substantially equal to, or shorter than, that of the frame. Preferably, graft 14 has a thickness of up to about 0.45 mm (preferably even thinner, up to about 0.2 mm and even up to about 0.11 mm). Optionally, sheet-like graft 14 is secured to an end of the frame. Further optionally, both ends of graft 14 are secured to the frame.

Graft 14 may optionally be of a material through which cells are able to proliferate, or alternatively, which is impervious to cell proliferation. The material may optionally be substantially impermeable to fluids, and/or a stretchable or collapsible material.

Graft 14 may optionally include at least one marker which is detectable by a medical imaging modality, such as radiation emission, X-ray transmission, magnetic resonance imaging and ultrasound. The marker may optionally be disposed on at least one of the edges or ends of the graft, and may further optionally delineate the edges or ends of the graft. In embodiments, an alignment hole is configured to function as a marker.

Useful materials from which to fashion a graft for use in any of the above embodiments of the present invention include synthetic or polymeric material including but not limited to polytetrafluoroethylene, urethane, elastomer, polyamide (e.g., Nylon) and polyester (e.g., Dacron).

Useful materials from which to fashion a graft of the above embodiments of the present invention are also biological tissue including but not limited to autologous tissue, heterologous tissue, venous tissue, arterial tissue, serous tissues, serous membranes pleura, peritoneum, pericardium, dura mater and aortic leaflet. Generally suitable tissue types include but are not limited to equine, porcine, bovine or human tissue. It is often preferred that the tissue be thinned, that is after harvesting one or more layers of the harvested tissue are removed, e.g. by scraping, shaving, slicing or skiving (see U.S. Pat. Nos. 6,468,300 and 6,254,627 of the Inventor). Preferably, the tissue is thinned to less than about 0.45 mm thick, preferably to between about 0.05 mm and about 0.20 mm.

In order to increase the toughness of the tissue, it is often advantageous to treat the tissue, for example with a glutaraldehyde or a phosphate solution, in order to cross-link collagen in the tissue.

An implantable graft assembly for deployment within cranial vessels preferably comprises a graft 14 fashioned from serous tissue or serous membrane. One readily available type of serous tissue is bovine pericardium, a material shown to resist suture line bleeding, require no pre-clotting, support endothelialization and have an excellent host-tissue response. Further, bovine pericardial tissue has an elasticity of up to about 10% which allows the tissue constituting the graft to conform to both the unexpanded and expanded configurations of the expandable frame without adding a great deal of bulk which increases the profile on the balloon used to deploy the graft assembly. A particularly preferred bovine pericardium has cross-linked collagen. Bovine pericardium tissue is available in a thickness ranging from about 0.25 mm to about 0.75 mm, with an average of about 0.45 mm. Thicknesses of 0.45 mm and less are preferred, so long as the mechanical strength remains sufficient. Other tissue suitable in the practice of the invention includes porcine pericardium, equine pericardium, heterologous peritoneum, heterologous pleura, aortic leaflet, dura mater and others. Useful tissue is relatively impenetrable, which prevents build up and the migration of smooth muscle cells through the stent frame.

Serous membranes are made of two strata. The serous stratum of a serous membrane is a very smooth single layer of flattened, nucleated mesothelial cells united at their edges by cement. The serous stratum rests on a basement layer. For some embodiments, natural serous membrane comprising both the serous stratum and the basement layer strong, elastic and thin enough to be useful in implementing the teachings of the present invention. However, in embodiments, a preferred material from which to fashion a graft 14 is thinned serous membranes where at least a portion and in embodiments all of the basement layer has been removed as taught in U.S. Pat. Nos. 6,254,627 and 6,468,300 of the. Not only is thinned serous membrane sufficiently strong, elastic and even thinner than serous membrane, thinned serous membrane also provides little resistance to radial expansion, making thinned serous membrane one of the few materials suitable for use in covering or jacketing self-expanding stents. Thus, in a preferred embodiment, the graft is substantially fashioned from a thinned serous membrane where a harvested serous membrane (dura mater, peritoneum, pericardium or pleural tissue especially porcine, bovine, equine and human serous tissue) has been processed by removal of a layer of at least some of the basement layer (and thus thinned), preferably removal of all the basement layer (by a variety of suitable methods including peeling, shaving or otherwise removing a thin layer of the basement layer), leaving only the serous stratum. In an embodiment of the present invention, a graft 14 is thinned serous membrane that is substantially the serous stratum of serous tissue devoid of basement layer. Preferably, a graft of the present invention has a thickness of between about 0.05 mm and about 0.20 mm.

In embodiments, a graft assembly of the present invention is made by providing a radially expandable substantially tubular frame, as described above, providing a graft (preferably a sheet-like graft) of the appropriate materials and dimensions, and c) associating (e.g., securing or attaching) the graft to the frame so that the graft covers only a portion of the surface area of the frame. In embodiments of the present invention the graft has two ends defining a length of the graft and two edges defining a width of the graft. In embodiments, the graft is rectangular.

In embodiments where it is desired to cover only a portion of the circumference of the frame associating the graft to the frame comprises associating the edges to the frame in such a way that between the two edges is a gap, generally through which is apparent a portion of the frame so that when the graft assembly is deployed the graft covers only a portion of the circumference of the frame.

In embodiments where it is desired to cover the entire circumference of the frame, associating the graft to the frame comprises associating the edges to the frame in such a way that the two edges abut (see FIG. 1a) or overlap so that when the graft assembly is deployed the graft covers an entire circumference of the frame.

In embodiments, the frame is substantially a stent and the graft is secured to the stent, for example as known in the art or as depicted in FIGS. 1D and 1E. In embodiments, distributed over the surface of the frame are a plurality of graft-connecting features (as depicted in embodiments of the stents taught in U.S. Pat. No. 6,929,658 of the Inventor) and securing the graft to the frame comprises securing the graft to at least one graft-connecting feature in accordance with dimensions of the graft (as depicted in FIGS. 1A, 1B and 1C).

In embodiments where the substantially tubular frame comprises two radially-expandable ring sections mutually connected with longitudinally arrayed struts as described above and depicted in FIGS. 2A, 2B, 2C, 2D, 2E and 2F, and securing the graft to the frame comprises securing the graft to locations selected from the group consisting of the ring sections, the struts and a combination thereof. In embodiments, a graft is secured to two struts but spanned over at least one intervening strut so that the graft roughly follows the contour of the (imaginary) tube which is defined by the frame. In embodiments, a graft is the full-length of the frame and is secured to two struts but lays over the outer surface of the expandable ring sections, so that when the assembly is expanded, the graft is stretched over the frame by the struts, but is forced to roughly follow the contour of tube which is defined by the frame by the expandable ring sections. In embodiments, along the length of at least two of the longitudinally arrayed struts are a plurality of graft-connecting features (e.g. as depicted in 2C and 2D) and securing the graft to the frame comprises securing the graft to at least one such graft-connecting feature in accordance with dimensions of the graft.

For example for the graft assembly depicted in FIG. 2B, where a graft 14 comprises a strip of tissue having a length equal to that of frame 20, graft 14 may be formed by cutting a rectangle of tissue having a length about equal to that of stent 20 and a width about equal to the portion of the circumference of the expanded stent to be covered. The portion of the circumference to be covered is the circumferential spacing between two struts 24, such that the two ends corresponding to the length of stent 20 may be associated to two struts 24, such as by sewing/suturing or other mechanical means such as staples, adhesive or chemical bonding and the like. The rectangle of tissue having a width about equal to the portion of the circumference of the expanded stent to be covered may be provided on the unexpanded stent in a folded or wrapped configuration. In one embodiment, the tissue on the unexpanded stent forms wings on either side of the stent that are folded about the stent, reducing the profile of the assembly, and unfolding upon expansion of the frame.

For an embodiment in which graft 14 is substantially shorter than frame 20, and which extends around the full circumference of frame 20, graft 14 may be formed by cutting a rectangle of tissue having a length equal to the portion of frame 20 to be covered, and a width about equal to the circumference of the frame in an expanded state. The two edges of the graft 14 may then be secured together (overlapping or abutting, see FIG. 1A), such as by sewing with 6-0 or 8-0 polypropylene sutures. Other means for securing the edges of graft 14 together include mechanical means such as staples, adhesive, chemical bonding and the like.

The implantable graft assembly of any of the embodiments of the present invention may be used in the treatment of an aneurysm, including an aneurysm located on a branched blood vessel. Optionally, the aneurysm is a cerebral aneurysm. Optionally, the aneurysm is a wide-necked aneurysm.

Aspects of the present invention relate to a method of treating an aneurysm by deploying an implantable graft assembly, preferably according to any of the embodiments of the present invention described above, within the blood vessel on which the aneurysm is situated, such that a portion of the implantable graft assembly on which the graft is located is positioned across the neck of the aneurysm to seal the aneurysm. If the blood vessel on which the aneurysm is located is a branched vessel, the portion of implantable graft assembly which is free of the graft is preferably positioned over the branch point.

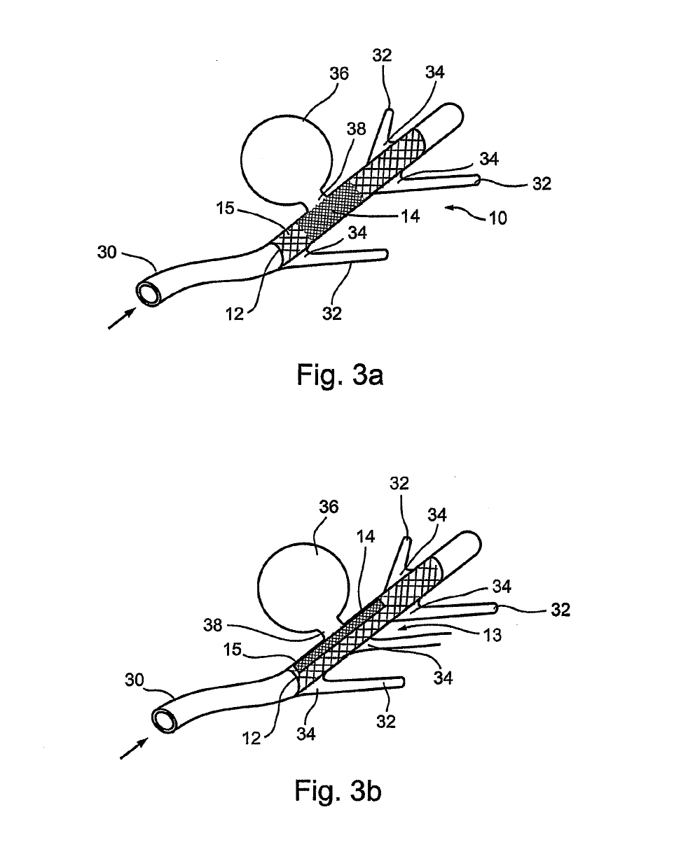

FIG. 3A depicts a bifurcated blood vessel with a trunk vessel 30, a plurality of branch vessels 32 and a plurality of bifurcation points 34. An aneurysm 36 is located on trunk vessel 30. An implantable graft assembly 10 having a graft 14 that is shorter than but covers the entire circumference of frame 12 (such as the graft assembly depicted in FIG. 1A) is shown in an expanded state within trunk vessel 30. Implantable graft assembly 10 is positioned within trunk vessel 30 such that graft 14 is positioned over neck 38 of aneurysm 36, and uncovered portion 15 is positioned over branch point 34, such that blood is able to flow through the struts of frame 12 into branch vessels 32.

Deployment of an implantable graft assembly of the present invention having a graft that covers the entire circumference of the respective frame (such as the depicted in FIG. 1A, 1D or 1E) is performed in a fashion analogous to the fashion in which prior art jacketed stents are deployed using a delivery catheter, for example with the help of a marker (on the frame, on the graft or on the delivery catheter) visible using a medical imaging modality.

FIG. 3B depicts a bifurcated blood vessel with a trunk vessel 30, a plurality of branch vessels 32 and a plurality of bifurcation points 34. An aneurysm 36 is located on trunk vessel 30. Implantable graft assembly 11 having a graft 14 that covers only 180° of the circumference of frame 12 (such as the graft assembly depicted in FIG. 1B, FIG. 1C or FIG. 2B) is shown in an expanded state within trunk vessel 30. Implantable graft assembly 10 is positioned within trunk vessel 30 such that graft 14 is positioned over neck 38 of aneurysm 36, and uncovered portion 15 is positioned over branch point 34, such that blood is able to flow through the struts of frame 12 into branch vessel 32.

Deployment of an implantable graft assembly of the present invention having a graft that covers only a portion of the circumference of the respective frame (such as depicted in FIG. 1B, 1C or 2B) requires that the graft be oriented properly over the neck of the aneurysm. To this end, it is required that a delivery system used for deploying such a graft assembly be configured to control the radial orientation of the graft within the blood vessel.

In embodiments, a delivery system used is configured to control the radial orientation of the graft by rotation of the implantable graft assembly when inside the body during the deployment process. Generally, such deployment follows conventional procedures. For example, a catheter guiding guide wire is backloaded into a delivery catheter having the implantable graft assembly loaded over an inflatable balloon or on a self-expanding stent delivery system. The delivery catheter and a catheter guiding guide wire are percutaneously introduced by means of a conventional Seldinger technique and a 6 to 10 French guiding catheter into the patient's arterial system. The catheter guiding guide-wire is advanced through the vasculature under fluoroscopic imaging until it crosses the treated region, specifically across the neck of the aneurysm. Then the delivery catheter is advanced over the catheter guiding guide wire until the implantable graft assembly is maneuvered into position at the desired location within the treated region. The delivery system is used to rotate the graft assembly to the desired position, across from the neck of an aneurysm. The balloon is inflated or the securing mechanism of the self-expanding stent is released to expand the frame of the graft assembly thereby pressing the graft against walls of the blood vessel and over the neck of the aneurysm so as to substantially seal the neck of the aneurysm. In embodiments, rotating is with reference to an observable marker (functionally associated with, for example, the graft, the frame, the delivery system), for example a marker observable by a medical imaging modality.

If applicable the balloon is then deflated, and the delivery catheter and catheter guiding guide wire removed, leaving the expanded implantable graft assembly in place, for example as depicted in FIG. 3B.

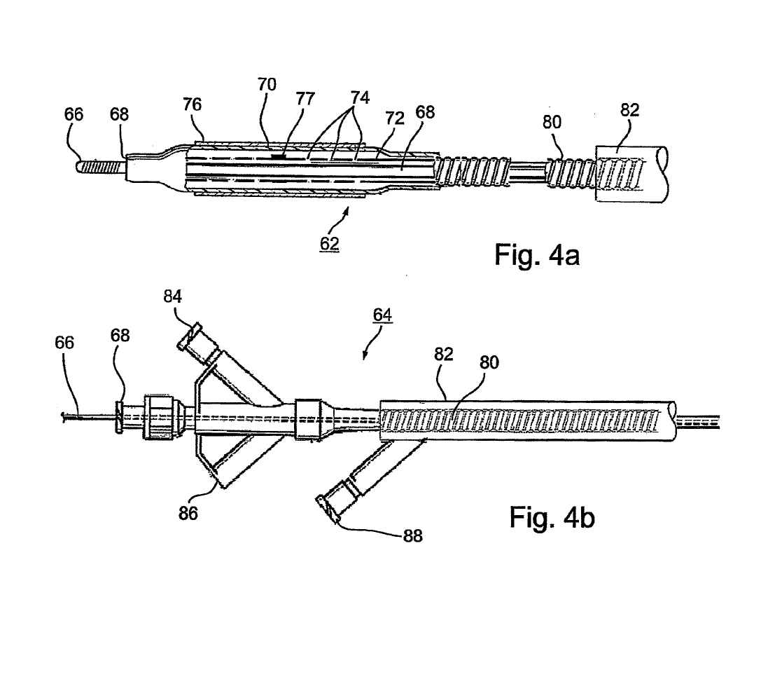

An embodiment of a delivery system configured to control the radial orientation of the graft by rotation of the implantable graft assembly when inside the body during the deployment process includes a delivery catheter such as depicted in FIG. 4A (distal end 62 of the delivery catheter) and FIG. 4B (proximal end 64 of the delivery catheter).

Distal end 62 of the delivery catheter is similar to that of prior art balloon catheters known in the art of stent delivery, and includes the distal end of a guide wire 66 running through a guide wire lumen 68, around which is arranged a stent-expanding balloon 70 in fluid communication with an inflation/deflation lumen 72 through inflation/deflation orifices 74. Graft-assembly 76 of the present invention (substantially similar to a graft assembly depicted in FIG. 1B, 1C or 2A) is crimped over balloon 70 so that the center of the graft (not depicted) of graft assembly 76 is over radio-opaque (and/or ultrasound opaque) marker 77. Proximal to balloon 70 is drive shaft 80 surrounded by external sleeve 82. Drive shaft 80 and external sleeve 82 are similar to corresponding components in the commercially available X-Sizer® Catheter System (ev3 corporation, Plymouth, Minn., USA) and allow rotation of drive shaft 80 inside sleeve 82 and consequently rotation of balloon 70 and graft assembly 76.

Proximal end 64 of the delivery catheter is similar to that of prior art balloon catheters, and includes the proximal end of guide wire 66 entering guide wire lumen 68. Opposing balloon inlation/deflation port 88 is rotation handle 86. In fluid communication with the lumen of sleeve 82 is external sleeve infusion port 88.

Deployment of graft assembly 76 using the delivery catheter depicted in FIGS. 4A and 4B is performed substantially as described above. Continuously, or only when it is desired to rotate drive shaft 80 or to purge air from the lumen of sleeve 82, a fluid such as heparinized saline is injected into port 84 and shaft rotated with the help of port 84 and rotation handle 86. The degree of rotation and accurate positioning of the graft is performed with reference to marker 77 observed with the help of an appropriate medical imaging modality.

In embodiments, a drive shaft is provided with a ferromagnetic portion along a carotid section of the drive shaft, that is a portion of the drive shaft that is located in the carotid artery during deployment of a graft assembly and a powerful adjustable magnet placed around the neck of the subject being treated. When the graft assembly is across the neck of the aneurysm, the adjustable magnet is used to apply a force (torque) to the ferromagnetic portion of the drive shaft, causing the drive shaft and consequently the graft to rotate. The adjustable magnet may be programmed to ensure accurate positioning of the graft across the neck of the aneurysm.

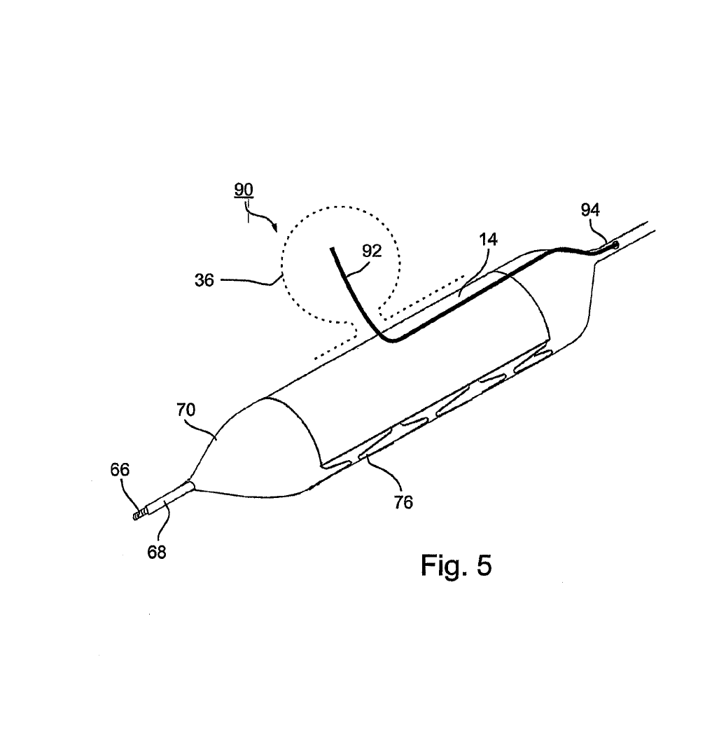

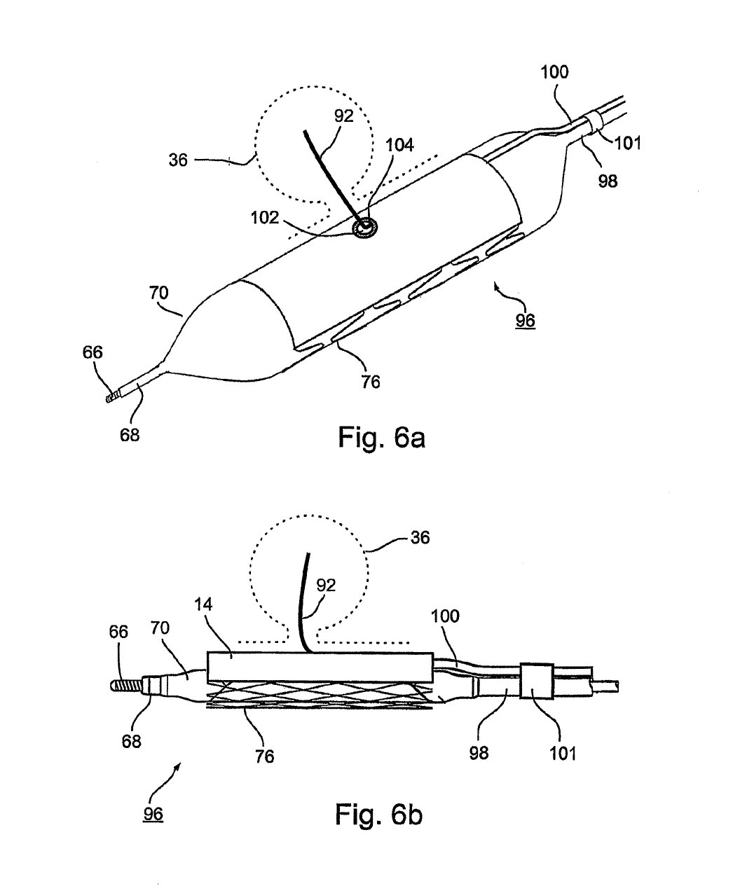

In embodiments, a delivery system is configured to control radial orientation of the graft with reference to an orientation guide wire 92 as depicted in FIG. 5 or in FIGS. 6A and 6B. In such embodiments, the delivery system comprises a catheter guiding guide wire 66, an orientation guide wire 92 and an appropriately modified delivery catheter. The delivery catheter includes a region near a distal end of the delivery catheter on which the implantable graft assembly is positionable for deployment (for example over a balloon for inflating the expandable frame of a graft assembly of the present invention, a first guide wire lumen for engaging the catheter guiding guide wire running from a proximal end of the delivery catheter through a distal end of the delivery catheter; a second orientation guide wire lumen for engaging the orientation guide wire running from a proximal end of the delivery catheter through a port in the side of the delivery catheter proximal to the region.

For deploying a graft assembly, the graft assembly is placed on the appropriate region of the delivery catheter so that the graft is in line with the port of the second lumen; the catheter guiding guide wire is placed in the blood vessel across the neck of the aneurysm and the orientation guide wire is placed in the blood vessel and into the aneurysm through the neck of the aneurysm. The delivery catheter with the graft assembly is mounted onto the two guide wires: the catheter guiding guide wire in the first lumen and the orientation guide wire in the second lumen.

By guiding the delivery catheter along the two guide wires, the graft is maneuvered to the proximity of the neck of the aneurysm along the catheter guiding guide wire and the orientation guide wire, thereby ensuring that the graft is aligned with the neck of the aneurysm. The catheter guiding guide wire passes through the entire delivery catheter from outside the patient all the way through the end of the delivery catheter, including through the region of the delivery catheter over which the graft assembly is located, analogously to guide wires known in the art of stent delivery. In contrast, the orientation guide wire passes through the delivery catheter and emerges proximally to the region of the delivery catheter over which the implantable graft assembly is located mounted on the same side where the graft is positioned and into the aneurysm through the neck of the aneurysm. As the delivery catheter progresses along the two guide wires, the entire delivery catheter is directed by the orientation guide wire in such a way that the graft is properly located with respect to the neck of the aneurysm.

When in place, the frame of the graft assembly is expanded, thereby pressing the graft against walls of the blood vessel and across the neck of the aneurysm so as to substantially seal the neck of the aneurysm.

A first embodiment of a delivery system of the present invention including two guide wires where the orientation guide wire passes over the outside of a graft assembly is depicted in FIG. 5. In FIG. 5 is depicted the distal end of delivery catheter 90. Delivery catheter 90 is similar to that of prior art balloon catheters known in the art of stent delivery, and includes the distal end of a catheter guiding guide wire 66 running through a guide wire lumen 68 from the proximal end (not depicted) of guide wire lumen 68 out through the distal end of guide wire lumen 68 at the distal end of catheter 90. Graft assembly 76 including a graft 14 is crimped over a balloon 70, balloon 70 configured to function in the usual way. Unlike prior art stent-delivery catheters, delivery catheter 90 includes an additional orientation guide wire lumen that runs from the proximal end of delivery catheter 90 (not depicted) to an orientation guide wire port 94 that is positioned proximally to balloon 70. In FIG. 5, an orientation guide wire 92 that passes through the orientation guide wire lumen of delivery catheter 90 is seen emerging from orientation guide wire port 94, passing over the outside of graft assembly 76 and into aneurysm 36.

Deployment of graft assembly 76 using delivery catheter 90 depicted in FIG. 5 is performed substantially as described above. As the distal end of orientation guide wire 92 is located inside aneurysm 36, orientation guide wire 92 forces guide wire port 94 and consequently also graft 14 to be oriented properly vis a vis aneurysm 36. Generally, orientation guide wire 92 is withdrawn from aneurysm 36 and away from balloon 70 prior to expanding of the frame of graft assembly 72 so as not to interfere with the expansion.

A second embodiment of a delivery system of the present invention including two guide wires where an orientation guide wire 92 passes between a delivery catheter 96 and a graft assembly 106 to emerge through an alignment hole 102 reinforced with gold grommet 104 penetrating through a graft 14 of graft assembly 76 is depicted in FIGS. 6A and 6B. FIGS. 6A and 6B depict the distal end of delivery catheter 96. Delivery catheter 96 is similar to that of prior art balloon catheters known in the art of stent delivery, and includes the distal end of a catheter guiding guide wire 66 running through a guide wire lumen 68 inside a main catheter shaft 98 from the proximal end (not depicted) of guide wire lumen 68 out through the distal end of guide wire lumen 68 at the distal end of delivery catheter 96. Graft assembly 76 including a graft 14 is crimped over a balloon 70, balloon 70 configured to function in the usual way.

Unlike prior art stent-delivery catheters, delivery catheter 96 includes an additional orientation guide wire shaft 100 that is substantially a tube that defines an orientation guide wire lumen. Orientation guide wire shaft 100 is secured to main catheter shaft 98 until close (point 101) to balloon 70, at which point orientation guide wire shaft 100 is allowed to dangle loosely on top of balloon 70 so as not to interfere with inflation of balloon 70. The distal end of orientation guide wire shaft 100 where the orientation guide wire lumen ends (not depicted) defines an orientation guide wire port that is hidden from view in FIGS. 6A and 6B underneath graft 14 of graft assembly 76. In FIGS. 6A and 6B, graft assembly 106 is crimped over balloon 70 and over orientation guide wire shaft 100 so that alignment hole 102 is substantially above the distal end of orientation guide wire shaft 100. In FIGS. 6A and 6B, an orientation guide wire 92 passes through the orientation guide wire lumen of orientation guide wire shaft 100 of delivery catheter 96 from the proximal end of orientation guide wire shaft (not depicted) to emerge from the orientation guide wire port through alignment hole 102 in graft 14 to pass into aneurysm 36.

Deployment of graft assembly 106 using delivery catheter 96 depicted in FIGS. 6A and 6B is performed substantially as described above. As the distal end of orientation guide wire 92 is located inside aneurysm 36, orientation guide wire 92 forces the guide wire port of guide wire shaft 100 and consequently also graft 14 to be oriented properly vis a vis aneurysm 36. Orientation guide wire 92 is withdrawn from aneurysm 36 either prior or subsequently to expanding of the tubular frame of graft assembly 106.

As is clear to one skilled in the art upon perusal of the description, the teachings of the present invention provide a general method that allows treating aneurysms, treating aneurysms on branched vessels, and treating cerebral aneurysms, by providing graft assemblies that are narrow and flexible, at least in part by the use of grafts that cover only a part of the surface of a substantially tubular expandable frame. Thus, the present invention provides a method of treating a cerebral aneurysm by deploying an implantable graft assembly within the vessel on which the aneurysm is located, thereby sealing the aneurysm as an alternative to stent-assisted coil placement.

As noted above, one of the greatest barriers to the use of covered stents and other graft-supporting expandable implants is that the grafts limit the flexibility of the implant and increase the profile of the implant to a degree that makes deployment in the cerebrovascular implant difficult if not impossible. It has been unexpectedly discovered that a graft made of serous tissue or membrane is strong enough, flexible enough and thin enough to overcome these problems and thus be suitable for use in a cerebrovascular implant, especially serous membrane thinned by removal of some or all of the basement layer provides a sufficiently thin, not bulky, flexible and tough material.

Thus, aspects of the present invention provide for the use of serous tissue (including, but not limited to, serous membrane devoid of at least a portion of associated basement tissue, and even all of the basement layer to leave substantially only the serous stratum) for the preparation of a cerebrovascular implant, for example as a graft component of an implantable graft assembly, for example an implantable graft assembly of the present invention. That said, it has been found that in embodiments the serous tissue as described above can also be used as a “prior art” stent cover for a stent that is thin and flexible enough, allowing deployment of the stent with the stent cover within the complex pathways of cranial blood vessels. Stents that are suitably thin and flexible and that can be covered with a stent cover made of serous tissue include the Over and Under™ stent (Design & Performance (Cyprus) Ltd.), Neuroform stent (Boston Scientific Corp. Natick, Mass., USA), Neurolink stent (Guidant, (Indianapolis, Ind., USA, now an affiliate of Boston Scientific Corp.)) or Boa stent (Balt, Montmorency, France).

In embodiments, a graft or stent cover is fashioned from serous tissue which has been thinned by removal of a layer of at least some of the basement layer, as discussed above. In embodiments, a graft is fashioned by removal of all the basement layer, leaving substantially only the serous stratum. The serous tissue is preferably derived from bovine pericardium, although in embodiments human, equine and porcine pericardium are used. Such a graft preferably has a thickness of between about 0.05 mm and about 0.20 mm, and most preferably between about 0.1 mm and about 0.15 mm.

Depending upon the material from which a graft is made, it may be required to maintain the graft wet at all times before deployment. The implantable graft assembly of any of the embodiments of the present invention may optionally further comprise a coating. Suitable coatings include, for example, anti-thrombogenic coatings, anti-angiogenic coatings, anti-coagulant coatings and active pharmaceutical ingredient delivering coatings.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, the present invention is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A method of treating an aneurysm, comprising:
a) providing an implantable graft assembly including a graft secured to a radially expandable substantially tubular frame, wherein in an expanded state a first portion of the surface area of said frame is covered by said graft and a second portion of the surface area of said frame is free of said graft, said first portion covering a circumferential section which is less than the entire circumference of said frame.
b) providing a delivery system for deploying said implantable graft assembly within a blood vessel on which the aneurysm is located, said delivery system including a catheter guiding guide wire, an orientation guide wire and a delivery catheter, said delivery catheter including:
a region near a distal end of said delivery catheter on which said implantable graft assembly is positionable for deployment;
a first lumen for engaging said catheter guiding guide wire running from a proximal end of said delivery catheter through a distal end of said delivery catheter; and
a second lumen for engaging said orientation guide wire running from a proximal end of said delivery catheter through a port in the side of said delivery catheter proximal to said region
c) deploying said implantable graft assembly within said blood vessel using said delivery system, such that said portion of said frame covered by said graft is positioned across a neck of the aneurysm, said deploying comprising:
i) placing said implantable graft assembly on said region of said delivery catheter so that said graft is substantially in line with said port of said second lumen;
ii) placing said delivery catheter guiding guide wire in said blood vessel across said neck of the aneurysm;
iii) placing said orientation guide wire in said blood vessel and entering the aneurysm through said neck of the aneurysm;
iv) mounting said delivery catheter onto said guide wires, said catheter guiding guide wire in said first lumen and said orientation guide wire in said second lumen;
v) maneuvering said implantable graft assembly to proximity of said neck of the aneurysm along said catheter guiding guide wire and said orientation guide wire, thereby ensuring that said graft is aligned with said neck of the aneurysm; and
vi) expanding said tubular frame;
thereby pressing said graft against walls of said blood vessel and across said neck of the aneurysm so as to substantially seal said neck of the aneurysm.

2. The method of claim 1, wherein during iv said orientation guide wire is placed on the outside of said graft; and said orientation guide wire is withdrawn from said aneurysm prior to said expanding of said tubular frame.

3. The method of claim 1, wherein during iv said orientation guide wire is placed through an alignment hole penetrating through said graft.

4. The method of claim 1, wherein said aneurysm is a cerebral aneurysm.

5. The method of claim 1, wherein said delivery system comprises a balloon catheter including an inflatable balloon at said region near said distal end of said delivery catheter on which said implantable graft assembly is positionable for deployment; and
said expanding said tubular frame includes inflating said inflatable balloon.

6. The method of claim 1, where said tubular frame is self-expanding; and
said expanding said tubular frame includes releasing constraints preventing said tubular frame from expanding.

7. The method of claim 1, wherein said first portion of the surface area of said frame covered by said graft constitutes no more than about 95% of the surface area of said frame;

8. The method of claim 7, said first portion constituting no more than about 80% of the surface area of said frame.

9. The method of claim 1, wherein said first portion of the surface area of said frame covered by said graft has a length less than about 75% of the total length of said frame.

10. The method of claim 1, wherein said first portion of the surface area of said frame covered by said graft covers a circumferential section which is less than the entire circumference of said frame.

11. The method of claim 10, said first portion covering a circumferential section comprising not more than about 330° of the entire circumference of said frame.

12. The method of claim 11, said first portion covering a circumferential section comprising not more than about 180° of the entire circumference of said frame.

13. The method of claim 1, wherein said frame is substantially a stent.

14. The method of claim 2, said graft being substantially sheet-like.

15. The method of claim 14, said graft having two ends defining a length of said graft and two edges defining a width of said graft.

16. The method of claim 15, wherein said length of said graft is substantially equal to the length of said frame.

17. The method of claim 15, wherein said length of said graft is substantially shorter than the length of said frame.

18. The method of claim 15, wherein an end of said graft is secured to an end of said frame.
